(19) United States
(12) Patent Application Publication
(10) Pub. No.: US 2004/0000648 A1
(43) Pub. Date: Jan. 1, 2004

(54) E-beam treatment system for machining coolants and lubricants

(76) Inventors: Lawrence D. Rissler, Sumner, WA (US); Daniel A. Hendricks, Kenmore, WA (US); Tzvi Avnery, Winchester, MA (US)

Correspondence Address: HARNESS, DICKEY & PIERCE, P.L.C., P.O. BOX 828, BLOOMFIELD HILLS, MI, 48303, US

(21) Appl. No.: 10/186,170

(22) Filed: Jun. 28, 2002

(51) Int. Cl. A61L 2/14

(52) U.S. Cl. 250/435; 250/492.3

(57) ABSTRACT

A treatment system and method for substantially reducing or eliminating bacteria in bacteria laden fluids used with machine tools. An electron beam emitter is used to irradiate the fluid with an electron beam. The electron beam creates an atmospheric plasma region that kills bacteria exposed to it. The fluid is thus decontaminated when circulated through the electron beam emitter. In one embodiment a filter is employed to remove particulates entrained in the fluid. A pump is also employed for withdrawing fluid from the sump of the machine tool. In another embodiment a plurality of electron beam emitters are disposed in longitudinal alignment with one another and closely adjacent a fluid carrying conduit to provide an intensified atmospheric plasma region through which the bacteria laden fluid flows. The system eliminates the need for unscheduled fluid changing due to bacterial laden fluid problems. The system also eliminates the problem of disposing of the fluid as well as the risk of the operator coming in contact with the bacteria laden fluid during the treatment process, plus the additional equipment cleaning to remove bacterial sludge residue.

### FIELD OF THE INVENTION

This invention relates to systems for treating bacteria laden coolants and lubricants (hereafter known as fluids) used with machine tools, and more particularly to a system and method employing E-beam irradiation of the fluids used with machine tools to significantly reduce the level of bacteria in such fluids or to eliminate such bacteria entirely from such fluids.

### BACKGROUND OF THE INVENTION

With present day machine tools, various cutting fluids are employed which act as coolants and/or lubricants for various components of the machine tool and/or the work piece(s) being machined by the machine tool. Over a period of time, these fluids become contaminated with bacteria and metal finds (swarf). When this happens, there are three common methods of handling these contaminated fluids: 1) filtering the metal finds plus foreign matter (way oils and hydraulic oils); 2) treating the bacteria with chemicals; and 3) disposing of the contaminated fluid and replacing it with new fluid. As can be appreciated, with present day environmental regulations, disposing of these types of machine tool fluids presents a rapidly growing disposal problem and must be carried out with care to ensure that damage to the environment does not occur. Also, the changing and replacement of fluids can represent a significant cost over a given length of time for a given machining process.

Various methods have also been employed in an attempt to treat bacteria laden fluids such that the fluid can be used over an extended prior period of time. These attempts, however, have introduced other drawbacks and concerns. One principal concern is the need for various chemicals to accomplish the removal of bacteria from the fluid being treated. The use and handling of various types of chemicals for this purpose is undesirable because, during the process of using such chemicals, highly trained individuals wearing PPE (Personal Protective Equipment) are required. Otherwise, this creates an extreme health and safety problem where the hands, arms or other areas of an individual may come in contact with hazardous chemicals. The application of such chemicals also raises the risk of the individual coming in contact with the bacteria laden fluid during the treatment process.

Still other disadvantages with present day fluid treatment systems for machine tool operations is the down-time requirement that may be introduced as a result of the treatment process. In many applications, it is very important that machine tools remain operating for extended periods of time. Changing the fluids, or even treating the fluids with chemicals, often requires that the machine tool being serviced be shut down until the fluid treatment, equipment sump cleaning, or fluid replacement process is completed. As can be appreciated, this is undesirable from a productivity and efficiency standpoint.

In view of the foregoing, there is a need to provide a fluid treatment system and method for treating bacterially laden fluids used with various forms of machine tools. More specifically, there is a need to provide a system and method for treating bacteria laden fluids being used with a machine tool without the requirement of turning off the machine tool while the fluid treatment is taking place. Still another important need is to be able to treat bacteria laden fluid such that the level of bacteria is significantly reduced or that the bacteria is completely eliminated from the fluid without requiring the user to handle or apply various hazardous chemicals to the fluid. Still another important need is to provide a system and method for treating bacteria laden fluids such that treatment occurs continuously or periodically while the fluid is being used with a given machine tool or component thereof.

### SUMMARY OF THE INVENTION

The above needs are met by a fluid treatment system and method that substantially reduces, or entirely eliminates, bacteria from a fluid used with a machine tool. The system employs an electron beam (“E-beam”) emitter which is disposed closely adjacent a fluid flow path of the fluid. The E-beam emitter generates a broad electron beam to create an atmospheric plasma region through which the fluid passes. The electron beam kills the bacteria entrained in the fluid as the fluid passes through the atmospheric plasma region.

In one preferred form the system and method of the present invention employs a pump for pumping fluid from a sump or reservoir of the machine tool. A filter is employed for filtering particulates entrained in the fluid. The filtered fluid exiting from the filter is then input into the E-beam apparatus where it is irradiated by the electron beam. The fluid is then directed via a conduit back to the sump of the machine tool.

In another preferred form, a plurality of E-beam apparatuses are disposed closely adjacent to a fluid carrying conduit associated with the machine tool. Each E-beam apparatus generates an atmospheric plasma region that extends through the interior of the conduit, chamber, or enclosure being irradiated. The fluid flowing through the conduit thus experiences a highly concentrated electron beam that serves to kill bacteria in the fluid as the fluid passes through the concentrated electron beam.

In both of the above-described embodiments, the level of bacteria is successively reduced each time the fluid passes through the electron beam. Thus, over a relatively short period of time, the level of bacteria can be reduced to a harmless level or virtually eliminated. In both embodiments, there is no need to shut down the machine tool while the fluid is being treated. Also importantly, there is no need for the operator to apply any chemicals to the fluid or to otherwise come in contact with the bacteria laden fluid while treatment of the fluid is occurring. The present invention thus forms a very efficient means for treating bacteria laden fluid and eliminates the disposal problems created with the unscheduled bacterial laden fluid changing and disposal of such fluids.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a diagram of a fluid treatment system in accordance with a preferred embodiment of the present invention; and

FIG. 2 is a simplified diagram of an alternative preferred embodiment of the present invention employing a plurality of E-beam emitters disposed closely adjacent an existing fluid flow conduit through which the bacteria laden fluid passes.

### DETAILED DESCRIPTION OF THE PREFERRED EMBODIMENTS

The following description of the preferred embodiment(s) is merely exemplary in nature and is in no way intended to limit the invention, its application, or uses.

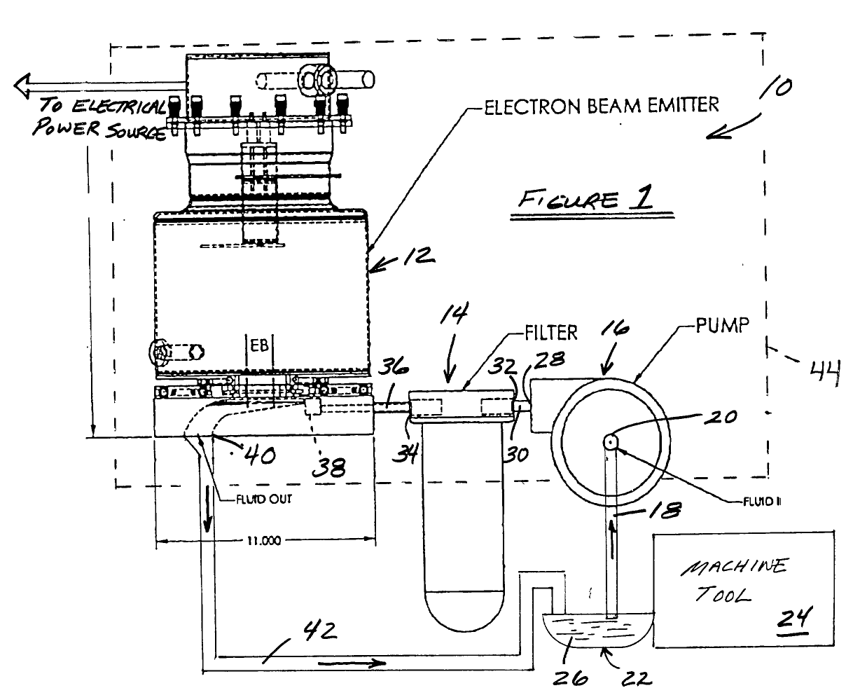

Referring to FIG. 1, there is shown an E-beam treatment system 10 in accordance with a preferred embodiment of the present invention. The system 10 generally includes an electron beam emitter 12, a filter 14 and a pump 16. A conduit 18 couples a fluid inlet 20 of the pump 16 with a sump 22 of a machine tool 24. The fluid 26 in the sump 22 is used for cooling and/or lubricating portions of the machine tool 24 or a work piece being machined by the machine tool. The fluid 26 becomes laden with bacteria over a given period of time if not treated or replaced.

The pump 16 includes a fluid outlet 28 that is coupled via conduit 30 to an inlet 32 of the filter 14. An outlet 34 of the filter 14 is coupled to a conduit 36 that is in turn coupled to a fluid inlet 38 of the electron beam emitter 12. The electron beam emitter 12 includes a fluid outlet port 40 that is coupled to a conduit 42. The conduit 42 extends back to the sump 22.

The electron beam emitter 12 is a commercially available component. One supplier is Advanced Electron Beams, Inc. of Wilmington, Mass. Briefly, the electron beam emitter 12 functions to create a broad electron beam which forms an atmospheric plasma region. Bacteria laden fluid flowing through this atmospheric plasma region is killed as it is irradiated. As a given quantity of fluid is circulated through the electron beam emitter 12 a number of times, the level of bacteria in the fluid is thus successively reduced to the point where the bacteria is either at a sufficiently low level that it does not present a health concern to individuals, or the bacteria is virtually eliminated.

The electron beam emitter 12 is powered by a suitable electrical power source such as a 120 volt AC or 240 AC power source. The electron beam emitter 12 creates the atmospheric plasma region by generating electrons that are emitted thermionically from a hot filament in a vacuum. The electrons are accelerated through a vacuum using a DC voltage onto a thin re-enforced titanium membrane. The electron beam is spread into a broad and uniform distribution by electron gun geometry. The electrons pass through the membrane and exit directly into the atmosphere forming a visible atmospheric plasma region containing three electrons, ions, radicals excited neutral and electromagnetic radiation including X-rays. Additional details of the construction and principles of the operation of the electron beam emitter 12 can be found in “Air Sterilization Using Modular Electron Beam Plasma”, by M. Koch et al, Environmental Science and Tech., p. 2946, Vol. 29, No. 12,1995, the disclosure of which is hereby incorporated by reference.

With further reference to FIG. 1, the filter 14 operates to remove particulates entrained in the bacteria laden fluid 26 as the fluid is circulated through the filter. The filter 14 may comprise any commercially available filter suitable for this purpose. The pump 16 may comprise any commercially available form of fluid pump. Since the capacity of the pump 16 determines the rate of flow of the bacteria laden fluid 26 through the electron beam emitter 12, it will be appreciated that a pump having a higher output will allow any given quantity of bacteria laden fluid to be treated more quickly.

In operation, the pump 16 serves to draw fluid 26 from the sump 22 through the conduit 18 and into fluid inlet 20. The fluid 26 is then expelled from outlet 28 into the conduit 30. It passes from the conduit 30 through the filter 14 where particulates (i.e., finds) are removed the fluid 26. The fluid 26 then passes through conduit 36 into the fluid inlet 38 of the electron beam emitter 12. As the fluid 26 is circulated through the electron beam emitter 12 it passes through the atmospheric plasma region created by the electron beam generated by the electron beam emitter 12 wherein bacteria in the fluid 26 is killed. The fluid 26 is then directed through the outlet 40, through conduit 42 and back into the sump 22. It will be appreciated, however, that a variety of schemes could be applied for withdrawing the bacteria laden fluid from the sump 22 and returning the fluid to the sump. The illustration of conduits 18 and 42 is meant only as one exemplary arrangement for withdrawing the fluid 26 from the sump 22 and returning the fluid to the sump.

In an alternative preferred form of the present invention, a frame 44 or other structure suitable for supporting the electron beam emitter 12, the filter 14 and the pump 16 may be employed. Use of the frame 44 allows the system 10 to be configured as a portable system that can be used on a number of different machine tools as needed. If the frame 44 is employed, it will be appreciated that suitable clamps or other fittings will need to be employed at the inlet 20 of the pump 16 and at the fluid outlet 40 of the electron beam emitter 12 to permit easy hook up and detachment of the system 10 from any given machine tool 24. If the frame 44 is employed, the system 10 can be used with a given machine tool 24 for a desired period of time as needed to reduce or eliminate the bacteria from the fluid in the sump of the machine tool, and then moved to a different machine tool for the same purpose. System 10 could thus be moved from one machine tool to another in accordance with a user determined schedule such that the fluid used with any given number of machine tools can be periodically treated to maintain the bacteria level in the fluid used with each machine tool at an acceptably low level.

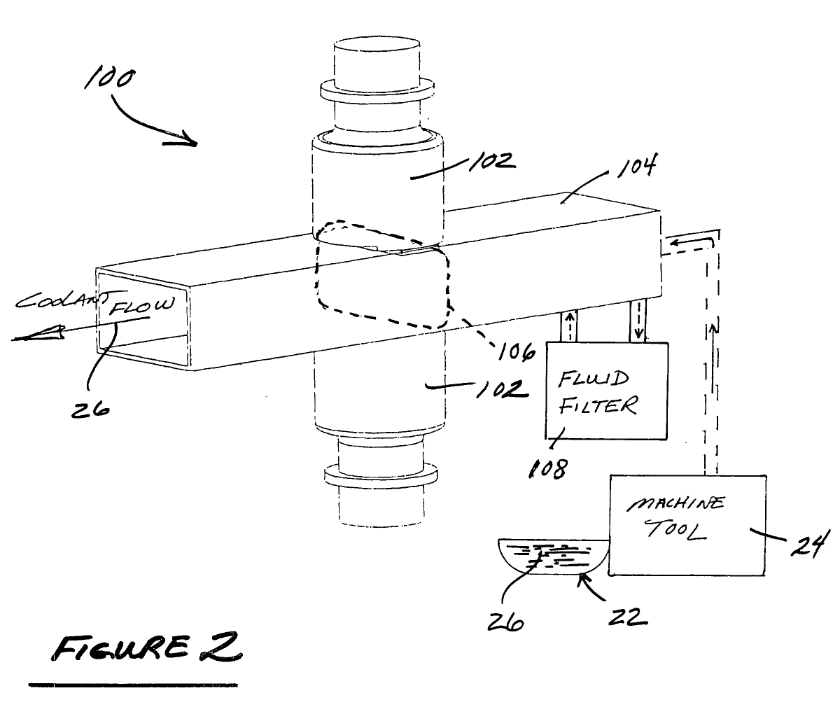

Referring to FIG. 2, an E-beam treatment system 100 in accordance with an alternative preferred embodiment of the present invention is illustrated. The system 100 generally employs a plurality of electron beam emitters 102 that are disposed closely adjacent a fluid carrying conduit 104. The conduit 104 may form an existing fluid conduit of a particular machine tool or may comprise a conduit that is interposed at a suitable location of a flow path through which the fluid used with a given machine tool passes during normal use. In either instance, the electron beam emitters 102 generate an intensified atmospheric plasma region 106 through which the fluid flowing within the conduit 104 passes during normal use of the machine tool. By placing the electron beam emitters in longitudinal alignment with one another, the highly concentrated atmospheric plasma region 106 is generated. It will be appreciated, however, that the system 100 forms a system that is dedicated to a given machine tool or possibly a given bank of machine tools. Optionally, a fluid filter 108 may also be included for filtering particulates entrained in the fluid withdrawn from the sump 22 of the machine tool 24.

The various preferred embodiments of the present invention thus provide a means for significantly reducing or eliminating bacteria from bacteria laden fluids used with a machine tool. This eliminates the need to frequently change the fluid and the attendant problems of disposing of the fluid, the cost associated with such fluid changes, and the possibility of user contact with the contaminated fluid. The present invention further eliminates the need for individuals to handle various hazardous chemicals that have previously been used in treating bacteria laden fluids.

Those skilled in the art can now appreciate from the foregoing description that the broad teachings of the present invention can be implemented in a variety of forms. Therefore, while this invention has been described in connection with particular examples thereof, the true scope of the invention should not be so limited since other modifications will become apparent to the skilled practitioner upon a study of the drawings, specification and following claims.

What is claimed is:

1. A system for reducing bacteria levels in a fluid used with a machine tool, said system comprising:
an electron beam emitter having a fluid inlet for receiving said fluid, said electron beam emitter operating to expose said fluid to an electron beam to reduce a level of said bacteria in said fluid and to output said fluid back to a portion of said machine tool.

2. The system of claim 1, further comprising a pump interposed between said electron beam emitter and said machine tool for pumping said fluid to said electron beam emitter.

3. The system of claim 1, further comprising a filter interposed between said machine tool and said electron beam emitter for removing particulates entrained in said fluid.

4. The system of claim 1, wherein said electron beam emitter operates to expose said fluid to an atmospheric plasma generated by said electron beam.

5. A system for reducing bacteria levels in a fluid used with a machine tool for lubricating or cooling a component of a machine tool or a work piece on which said machine tool is operating, said system comprising:
a pump for pumping said fluid from a sump of said machine tool;
a filter for receiving an output from said pump and filtering particulates from said fluid; and
an electron beam emitter having a fluid inlet for receiving said fluid from an output of said filter, said electron beam emitter operating to expose said fluid to an atmospheric plasma emitted by an electron beam to thereby reduce a level of said bacteria in said fluid, and to output said fluid back to said sump of said machine tool.

6. The system of claim 5, wherein said system comprises a portable system adapted to be readily moved from a first machine tool to a second machine tool.

7. The system of claim 5, wherein the pump includes a fluid supply tube for transferring said fluid from said sump to an inlet of said pump.

8. The system of claim 5, further comprising a conduit coupled to said output of said filter and to said fluid inlet of said electron beam emitter for supplying said fluid to said electron beam emitter.

9. The system of claim 5, further comprising a conduit coupled to an output of said electron beam emitter for transferring said fluid back to said sump.

10. A system for reducing bacteria levels in a fluid used with a machine tool for lubricating or cooling a component of a machine tool or a work piece on which said machine tool is operating, said system comprising:
a conduit arranged to receive said fluid as said fluid is pumped from a sump of said machine tool; and
at least one electron beam emitter disposed fixedly relative to said conduit, said electron beam emitter operating to expose said fluid to an atmospheric plasma emitted by an electron beam to reduce a level of said bacteria in said fluid as said fluid flows through said conduit and past said electron beam emitter.

11. The system of claim 10, further comprising a pair of said electron beam emitters disposed fixedly relative to said conduit for generating a pair of electron beams to which said fluid is exposed.

12. The system of claim 11, wherein said electron beam emitters are secured fixedly to said conduit.

13. The system of claim 12, wherein said electron beam emitters are secured in longitudinal alignment with one another.

14. The system of claim 13, wherein said electron beam emitters are secured so as to extend from opposite surfaces of said system.

15. A method for reducing bacteria levels in a fluid used with a machine tool for lubricating or cooling a component of a machine tool or a work piece on which said machine tool is operating, said method comprising:
directing said fluid to flow in proximity to an electron beam emitter; and
using said electron beam emitter to irradiate said fluid with an electron beam emitted by said electron beam emitter, said electron beam generating an atmospheric plasma region that operates to reduce a level of bacteria in said fluid upon exposure to said electron beam.

16. The method of claim 15, further comprising:
using a pump to pump said fluid from a sump of said machine tool into proximity with said electron beam emitter.

17. The method of claim 15, further comprising using a filter to filter particulates entrained in said fluid.

18. The method of claim 15, further comprising:
directing said fluid through a conduit; and
using a pair of said electron beam emitters disposed adjacent said conduit to irradiate said fluid with an intensified electron beam.

19. The method of claim 18, further comprising the step of disposing said electron beam emitters directly on said conduit.

20. The method of claim 18, further comprising said electron beam emitters in longitudinal alignment with one another on opposite sides of said conduit.
